(19) United States
(12) Patent Application Publication
(10) Pub. No.: US 2003/0073934 A1
(43) Pub. Date: Apr. 17, 2003

(54) Double slotted-cannula device and method of use

(76) Inventor: David A. Putz, Franksville, WI (US)

Correspondence Address: James P. Delaney, Jansson, Shupe & Munger, Ltd., Suite M, 245 Main Street, Racine, WI, 53403, US

(21) Appl. No.: 09/981,351

(22) Filed: Oct. 17, 2001

(51) Int. Cl. A61B 5/00; A61M 25/00

(52) U.S. Cl. 600/585

(57) ABSTRACT

An improved apparatus and method for accurately and safely implanting a probe into a brain including use of two cannulas having side-gaps extending longitudinally and opening into cavities. During implantation, the second cannula is positioned in the cavity of the first cannula such that the side-gaps are not juxtaposed, thereby defining a passageway through which the probe is inserted into the brain. Because the passageway is circumferential enclosed, the probe cannot veer into unintended brain tissue and is precisely positioned in the brain. The cannulas are then rotated relative to one another so that they may be separated from the probe either during or after withdrawal from the brain.

### FIELD OF THE INVENTION

This invention is related generally to intracranial implantation devices and, more particularly, to intracranial probe implantation devices for use during neurosurgery procedures.

### BACKGROUND OF INVENTION

The introduction into the brain of probes or other similar devices is common in many surgical procedures today. Such probes may include electrical, chemical, electrochemical, temperature and/or pressure sensors which enable the observation and analysis of the brain state. Typically, these sensors must be positioned at a specific point or region in the brain. Probes may also include sites from which treatment is provided to the brain. Such sites include ports for drug delivery or contacts which can transfer heat away from or to portions of the brain. These sites must also be positioned at specific points or regions in the brain.

Intracranial probes are typically fabricated so that their introduction to the brain is as minimally traumatic as possible. In addition to being minimally traumatic during implantation, probes must also be able to remain implanted without causing injury through unintended movement. In some uses, a probe may be implanted and remain in the patient's brain for weeks or longer. Changes in the positioning of the probe often occur during placement or during such extended periods. Therefore, the probe must be capable of precise placement and as biocompatible as possible. In response to these requirements, state of the art intracranial probes are typically thin, flexible pieces with smooth surfaces to minimize the amount of brain tissue contacted and to minimize damage to contacted brain tissue.

While such thin, flexible probes are sufficiently biocompatible, they are delicate and often difficult to insert along specific trajectories or lines of insertion. During typical implantation, a surgeon feeds the probe into the brain through an aperture in the skull. In this process, the surgeon has very little control over the distal end of the probe. In order to provide more rigidity to the probe to overcome this problem, a removable stylet may be inserted into the probe before implantation. Such stylets allow for somewhat more accurate intracranial positioning of the probe, however, they add limitations to the internal construction of the probe and may limit the probe's function. In addition, veering from the intended line of insertion is not altogether prevented by introduction of a stylet to the probe.

U.S. Pat. No. 5,116,345 to Jewell attempted to overcome the problems associated with accurate intracranial implantation through use of an apparatus including a skull plate screwed to the patient's skull, a shaft extending from the skull plate, an arc member attached to the shaft, a support having a bore attached to the arc member and a cannula inserted into a cannula holder positioned in the bore. This apparatus was intended to provide for both the drilling through the skull and the insertion of the cannula into the brain.

The Jewell patent discloses that a stylet may be positioned in the cannula to stiffen it and to close the axial opening during its insertion. After insertion of the cannula into the brain, the stylet is removed, thus revealing the axial opening. The probe is then inserted into the brain through the axial opening. The Jewell patent discusses removal of the cannula from the probe by moving the cannula laterally. It is disclosed that the cannula has a slot opening along one side which is wide enough that the probe can pass outwardly through it. Therefore, during implantation the probe is not prevented by the cannula from veering from the intended trajectory, nor from contacting brain tissue through the slot opening even if the probe remains in the cannula.

While typical intracranial probes have smooth surfaces so as to not cut any contacted tissue, many such probes are made of elastomers or other such materials which, although smooth, do not easily slide through brain tissue. The drag encountered by these types of probes can result in injury to the contacted brain tissue. When such probes are used with the Jewell cannula device, the presence of the exposed brain tissue through the slot opening, coupled with the probes' drag, causes the probe to veer unhindered into unintended areas of the brain.

Furthermore, because brain tissue may partially enter the cavity of the Jewell cannula after the stylet is removed, contact with even drag-less probes is likely. This contact may cause injury to the patient regardless of the probe's tendency to veer out of the cannula.

Therefore, there is a continuing significant need in the field of intracranial implantation, particularly with implantation of probes into the interior of the brain, for improvements in accuracy of implantation and avoidance of injury, while retaining efficiency and ease of use.

### OBJECTS OF THE INVENTION

It is an object of the invention to provide an improved intracranial implantation device which prevents injury to the patient.

Another object of the invention is to provide an intracranial implantation device which is simple in structure and operation in order to facilitate neurosurgery procedures.

Another object of the invention is to provide an intracranial implantation device which allows for precise implantation of electrodes in the brain while avoiding extensive trauma to and scarring of brain tissue.

Another object of the invention is to provide an intracranial implantation device including a slotted cannula received in another slotted cannula to provide a passageway for a probe to be inserted into a patient's brain.

Another object of the invention to provide a method of accurately implanting a probe in a patient's brain which minimizes injury to the brain.

Still another object of the invention is to provide a method of safely implanting a probe in a patient's brain through use of an assembly including two slotted cannulas.

These and other objects of the invention will be apparent from the following descriptions and from the drawings.

### SUMMARY OF THE INVENTION

This invention is an improved apparatus for accurately implanting a probe, such as an electrode, into a patient's brain and methods of use thereof. The invention represents a significant advance over the state of the art by providing novel elements, including an enclosed removable passageway through which a probe may be inserted into the brain.

The apparatus includes a guide assembly comprised of first and second cannulas. The assembly has distal and proximal portions with apertures at each end. The first cannula has a side-gap which extends longitudinally along its length and opens to a cavity. The second cannula is positioned within the cavity of the first cannula and has its own side-gap which extends longitudinally along its length and opens to a cavity. Each side-gap is dimensioned to be slightly larger than the probe so that the probe can be removed from either cannula. All components of the apparatus have smooth surfaces so as to prevent damage to contacted brain tissue.

The cannulas are arranged in a first orientation for implantation in which the side-gaps are not juxtaposed. This orientation provides for an enclosed passageway within the assembly. The passageway extends from the distal aperture to the proximal aperture.

The cannulas are rotatable with respect to one another. The cannulas can be rotated to a second orientation for removal in which the side-gaps are juxtaposed and define an assembly side-gap. The assembly side-gap is slightly larger than the probe so that the probe can be removed from the assembly.

The assembly may further comprise an obturator which is positioned within the passageway. The obturator has sufficient size to substantially fill the passageway so that upon insertion of the assembly into the brain, no brain tissue enters the passageway. The obturator has a smooth distal end which prevents harm to the brain upon insertion of the assembly into the brain. The obturator also includes an elastomer O-ring at its proximal end which secures the obturator to the cannula assembly to prevent the obturator from being dislodged from the assembly.

The method of use of the novel apparatus begins with preparing the assembly so that the cannulas are in the first implantation orientation. The obturator can be positioned in the passageway. The assembly is inserted, typically with use of stereotactic devices, into the brain through a bore in the skull along a predetermined trajectory or line of insertion. The assembly may be inserted so that it extends to the targeted portion of the brain, or it may remain short of the targeted portion of the brain. A proximal portion of the assembly remains outside the skull and can be manipulated by the surgeon.

The obturator is removed from the assembly through the assembly's proximal aperture. The probe is then inserted into the passageway through the assembly's proximal aperture such that the distal segment of the probe enters the brain while the proximal segment of the probe remains outside the brain. The probe may be inserted so that its distal end remains within the passageway or it may extend slightly beyond the assembly.

Once the probe is in its intended position, the cannula assembly is removed from the brain. This removal can be accomplished in several ways. In the preferred first method, the cannulas are rotated with respect to each other so that the assembly is in a second orientation for removal. In this orientation, the side-gaps of the cannulas are juxtaposed so that the entire assembly has a side-gap. The proximal segment of the probe is then moved through the exposed side-gap of the proximal portion of the assembly. The proximal portion is held in position while the assembly is withdrawn from the brain so that the probe passes through the entire length of the side-gap of the distal portion. In this manner, the assembly is withdrawn from the brain simultaneous with its separation from the probe.

A second method of removal of the assembly from the brain involves rotating the cannulas so that the assembly is in the second orientation for removal. The assembly is then withdrawn from the brain before the assembly is separated from the proximal segment of the probe by passing the proximal segment through the assembly side-gap during relative laterally movement between the probe and assembly. In this manner, the assembly is withdrawn from the brain before it is separated from the probe.

A third method of removal of the assembly from the brain involves removing the cannulas separately. The first cannula is withdrawn from the brain first and is separated from the probe by passing the proximal portion of the probe through the first side-gap as in the first or second methods. The second cannula is then withdrawn from the brain and separated from the probe in the same manner. The cannulas may or may not be rotated to the second removal orientation in this method.

### BRIEF DESCRIPTION OF THE DRAWINGS

FIG. 1 is an prospective view of the assembly in accordance with the principles of the present invention.

FIG. 2 is a cross section view of the assembly in implantation orientation in accordance with the principles of the present invention.

FIG. 3 is a cross section view of the assembly in implantation orientation with the obturator positioned in the passageway in accordance with the principles of the present invention.

FIG. 4 is a cross section view of the assembly in implantation orientation after the obturator has been removed and the probe has been inserted into the passageway in accordance with the principles of the present invention.

FIG. 5 is a cross section view of the assembly in removal orientation with the probe positioned in the passageway in accordance with the principles of the present invention.

FIG. 6 is a prospective view of the proximal portion of the assembly in accordance with the principles of the present invention.

FIG. 7 is another prospective view of the proximal portion of the assembly in accordance with the principles of the present invention.

FIG. 8 is a side view of the preferred obturator in accordance with the principles of the present invention.

FIG. 9 is a prospective view of the insertion of the distal portion of the assembly into the patient's skull in accordance with the principles of the present invention.

FIG. 10 is a prospective view of the removal of the obturator from the assembly in accordance with the principles of the present invention.

FIG. 11 is a prospective view of the insertion of the distal segment of the probe into the patient's brain through the passageway in accordance with the principles of the present invention.

FIG. 12 is a prospective view of the relative rotation of the first and second cannulas to define the assembly side-gap in accordance with the principles of the present invention.

FIG. 13 is a prospective view of the proximal segment of the probe having been moved through the assembly side-gap to allow for simultaneous separation of the assembly from the probe and withdraw of the assembly from the skull in accordance with the principles of the present invention.

FIG. 14 is a prospective view of the probe implanted in the patient's skull after the assembly is removed in accordance with the principles of the present invention.

### DETAILED DESCRIPTION OF THE PREFERRED EMBODIMENT

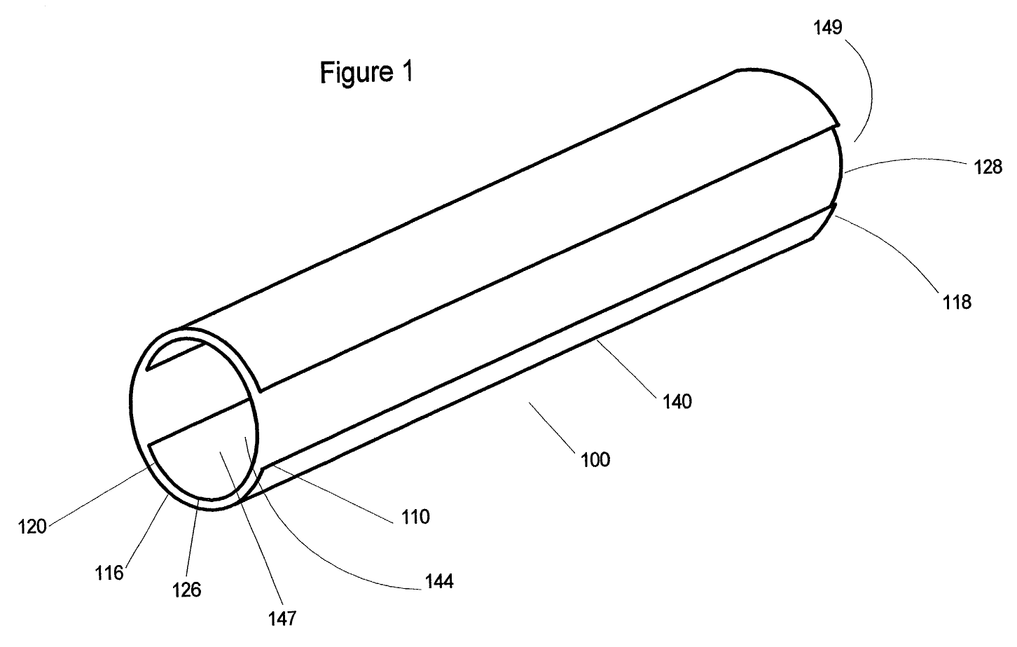

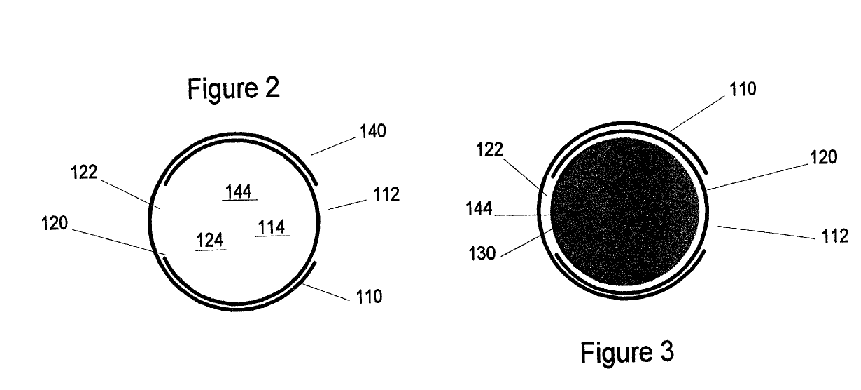

FIG. 1 is a prospective view of the double slotted cannula 100 in accordance with the invention. Assembly 140 comprises a first cannula 110, second cannula 120 and, optionally, an obturator (not shown). First cannula 110 has a side-gap 112 which leads to an internal cavity 114 as shown in FIG. 2. Side-gap 112 and cavity 114 extend from the first cannula's proximal end 116 to its distal end 118. Side-gap 112 and cavity 114 are dimensioned so that a probe may be inserted into cavity 114 through proximal end 116 and removed from cavity 114 through side-gap 112.

Second cannula 120 has a side-gap 122 which leads to an internal cavity 124 as shown in FIG. 2. Side-gap 122 and cavity 124 extend from the second cannula's proximal end 126 to its distal end 128. Side-gap 122 and cavity 124 are dimensioned so that a probe inserted into cavity 124 through proximal end 126 can be removed through side-gap 122.

The positioning of second cannula 120 in cavity 114 of first cannula 110 creates a passageway 144 through assembly 140. In the first implantation orientation, first side-gap 112 and second side-gap 122 are positioned non-juxtaposed so that the assembly defines an enclosed passageway 144. An obturator is inserted into passageway 144 so that when assembly 140 is inserted into a patient's brain during a procedure, no brain tissue enters passageway 144. After assembly 140 is inserted into the brain to the desired position for the probe the obturator is removed by pulling it through proximal aperture 147 at proximal ends 116,126 of cannulas 110,120.

FIG. 3 is a cross sectional view of the cannula assembly after the obturator has been inserted into the passageway. Obturator 130 substantially fills passageway 144 which is defined by the inner surfaces of first cannula 110 and second cannula 120. Obturator 130 preferably extends to distal aperture 149 so that no brain tissue enters passageway 144 when the distal portion of assembly 140 is inserted into the brain.

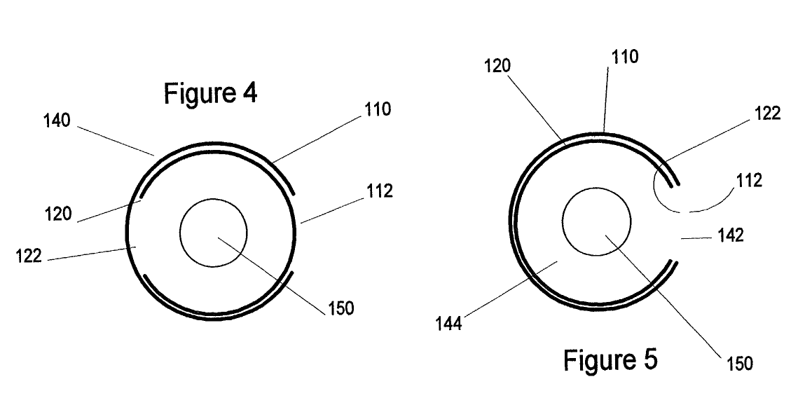

FIG. 4 is a cross sectional view of the cannula assembly after obturator 130 has been removed and probe 150 has been inserted. Probe 150 is preferably a depth electrode and has a diameter less than the width of first side-gap 112 and second side-gap 122. Therefore, probe 150 may be removed from first cannula 110 through first side-gap 112 and from second cannula 120 through second side-gap 122.

FIG. 5 is a cross section view of the cannula assembly after the first cannula 110 and second cannula 120 have been rotated relative to one another so that first side-gap 112 is juxtaposed with second side-gap 122 to define assembly side-gap 142. This positioning defines the assembly removal orientation. Probe 150 is able to pass out of passageway 144 through assembly side-gap 142 to enable removal of probe 150 from assembly 140.

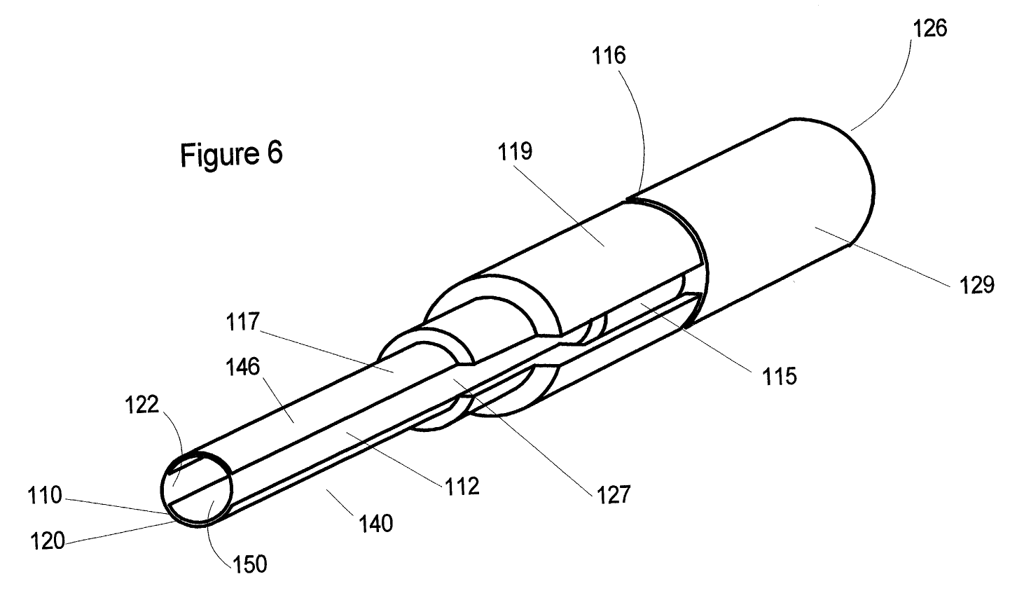

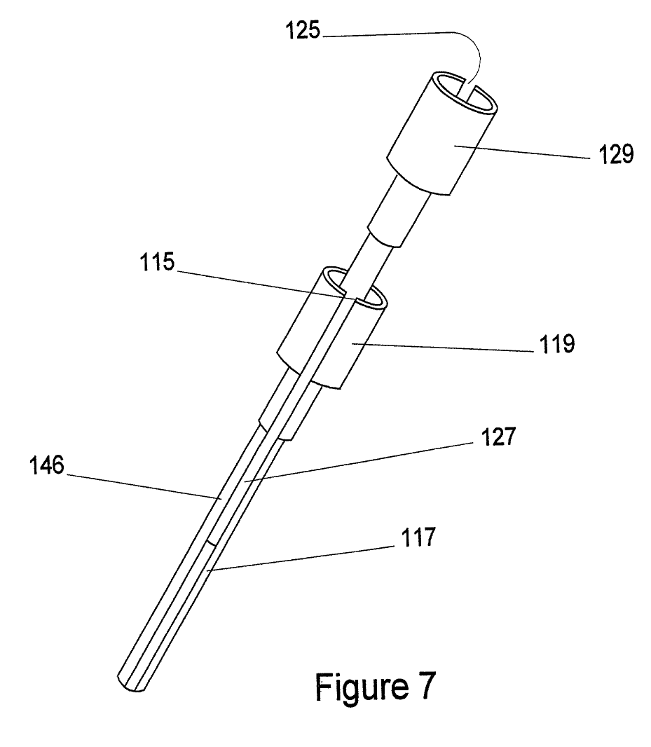

FIGS. 6 and 7 are prospective views of the preferred proximal portion of assembly 140. Proximal portion 146 of assembly 140 comprises first proximal portion 117 and second proximal portion 127. First proximal portion 117 includes first flanged end portion 119 which includes first annular recess 115. When second cannula 120 is inserted through first proximal end 116, first annular recess 115 receives second flanged end portion 129 so that first cannula 110 and second cannula 120 are fitted together and can be easily manipulated by a surgeon. First side-gap 112 extends through first flanged end portion 119 so that a probe may be removed through first side-gap 112. Second side-gap 122 extends through second flanged end portion 129 so that a probe may be removed through second side-gap 122. Before insertion into the brain, an obturator may be inserted through second proximal end 126. Second flanged end portion 129 includes a second annular recess 125 which is dimensioned to received a flanged end portion of obturator 130.

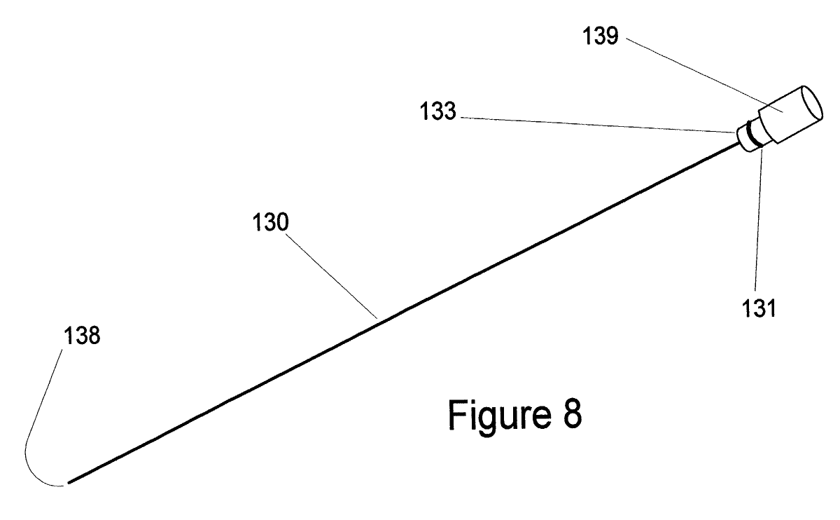

FIG. 8 is a side view of the preferred obturator. Obturator 130 includes flanged end portion 139 which fits into second annular recess 125. Located on flanged end portion 139 is O-ring or gasket 131 which is preferably a compressible and gummy material such as an elastomer. When obturator 130 is positioned in second annular recess 125, elastomer O-ring 131 provides sufficient friction so that obturator 130 does not accidentally fall out. However, even with the presence of elastomer O-ring 131, obturator 130 is easily removed from assembly 140 by the surgeon.

The length between distal end 138 and shoulder 133 is preferably equal to, or slightly greater than the distance between second annular recess 125 and distal aperture 149 so that obturator 130 prevents brain tissue from entering passageway 144. Distal end 138 of obturator 130 is preferably rounded to minimize injury to brain tissue it passes through. Obturator 130 has sufficient girth to substantially fill passageway 144 of assembly 140 so that no brain tissue enters passageway 144 during insertion into the brain.

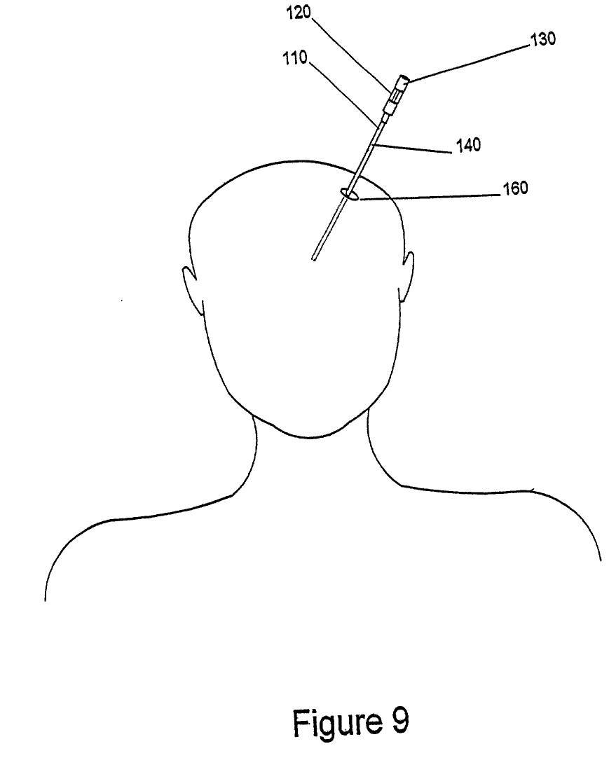

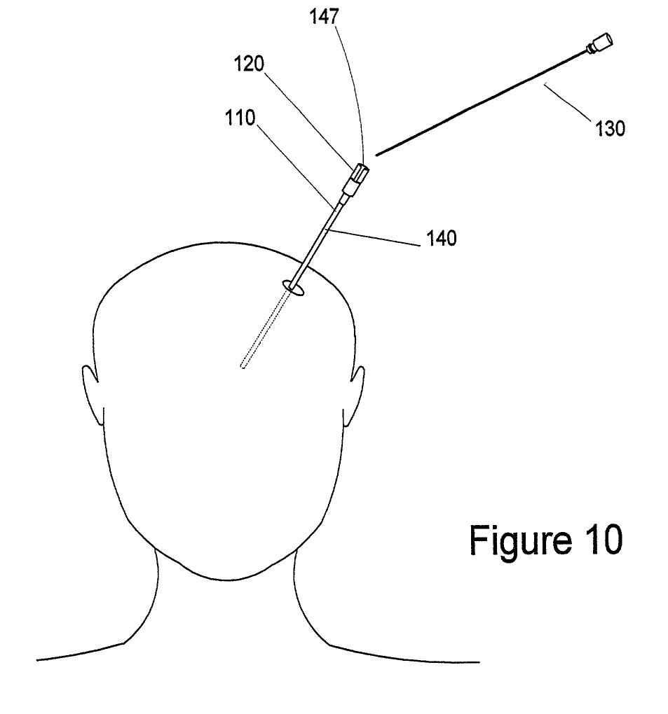

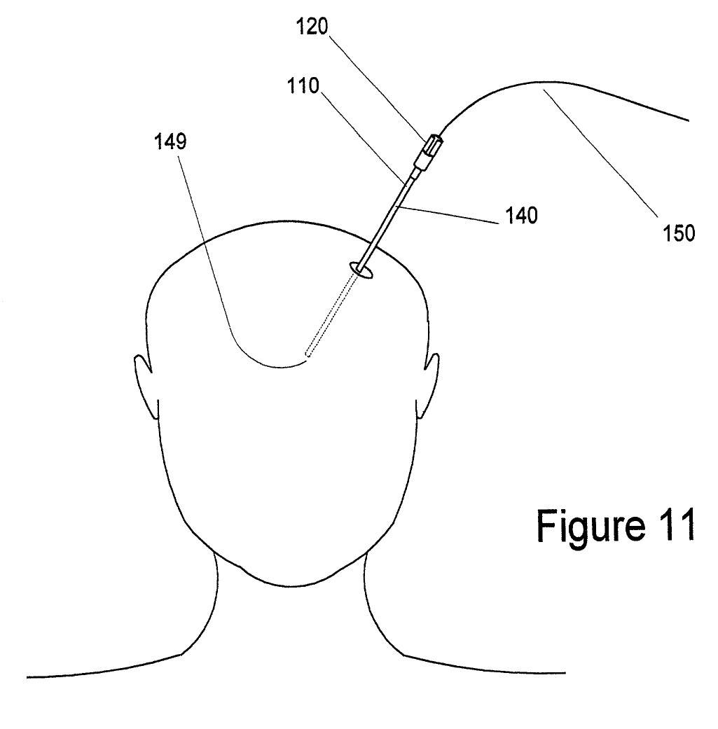

FIGS. 9-11 depict the implantation of probe 150 into the patient's brain. FIG. 9 shows assembly 140 having been inserted into the patient's brain through bore 160 drilled in the skull. Assembly 140 includes first cannula 110 and second cannula 120 with obturator 130 positioned in the passageway created by the cavities of the cannulas. FIG. 10 shows obturator 130 removed from assembly 140 through proximal aperture 147. FIG. 11 depicts probe 150 having been inserted into the passageway of the assembly through proximal aperture 147. The dashed lines demonstrate that probe passes though assembly 140 and extends slightly beyond distal aperture 149.

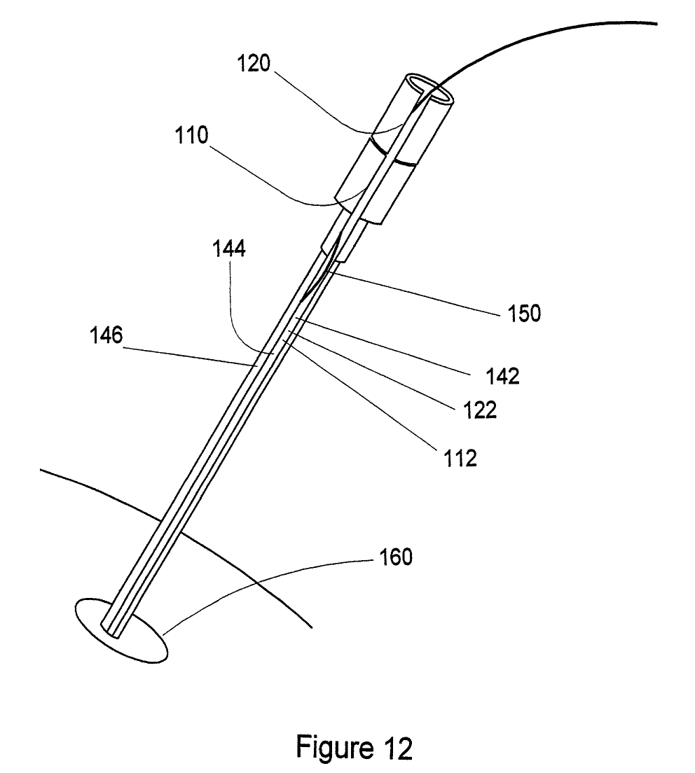

FIGS. 12-14 depict the preferred method of removing assembly 140 from the brain. FIG. 12 shows the alignment of first side-gap 112 with second side-gap 122 to create assembly side-gap 142. Probe 150 is positioned within passageway 144 and is dimensioned so that it can easily pass through assembly side-gap 142.

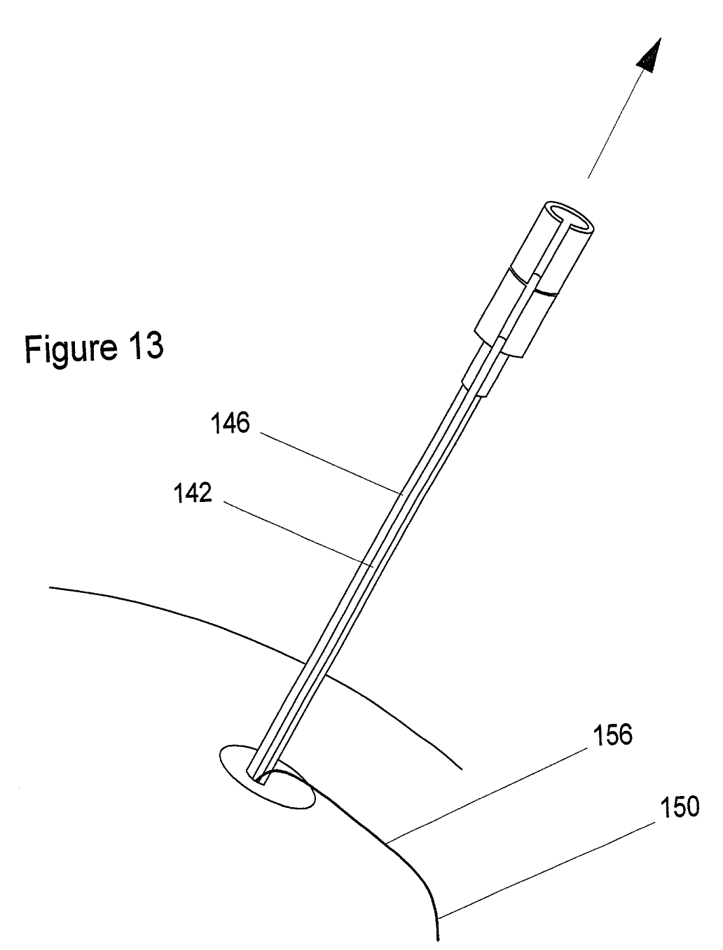

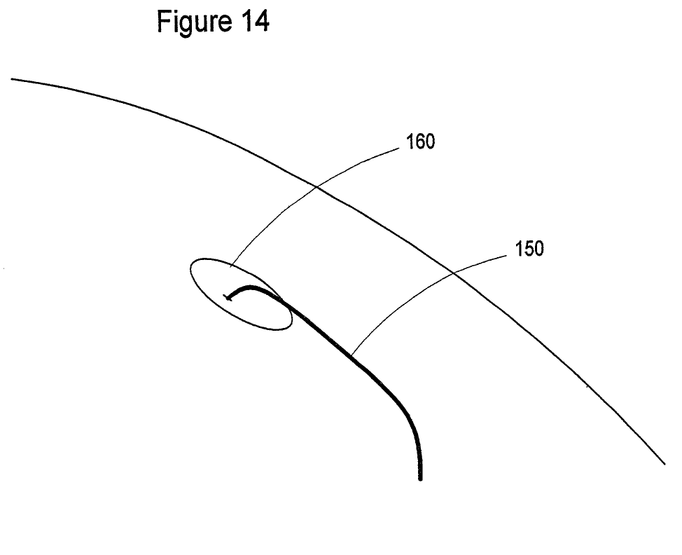

FIG. 13 depicts probe 150 pulled through assembly side-gap 142 in proximal portion 146 of assembly 140. The arrow denotes the direction in which assembly 140 is pulled out of the skull. When assembly 140 passes out of the patient's skull probe 150 remains in position, thereby passing through assembly side-gap 142 and distal aperture 149 of the previously implanted portion of assembly 140. FIG. 14 shows probe 150 implanted in the patient's brain through bore 160 after assembly 140 has been removed.

Thus, it should be apparent that there has been provided, in accordance with the present invention, an assembly for accurately and safely implanting a probe into a patient's brain that fully satisfies the objectives and advantages set forth above.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for accurately positioning a probe in a patient's brain comprising the steps of:
a preparing a guide assembly having distal and proximal portions and including a first cannula, second cannula and obturator, the first cannula having a first side-gap opening to a first cavity, the second cannula having a second side-gap opening to a second cavity, the second cannula positioned within the first cavity to define an enclosed passageway having a proximal aperture and the obturator positioned in the passageway;
inserting the distal portion of the assembly into the brain, such that the proximal portion remains outside the brain;
removing the obturator from the passageway;
inserting a distal segment of the probe into the brain through the passageway such that a proximal segment of the probe remains outside the brain;
rotating one of the cannulas with respect to the other cannula so that the first and second side-gaps are juxtaposed, thereby defining an assembly side-gap;
separating the assembly from the probe; and
withdrawing the assembly from the brain.

2. The method of claim 1 wherein the probe is a depth-electrode.

3. The method of claim 1 wherein the probe is inserted into the passageway through the proximal aperture.

4. The method of claim 1 wherein the obturator includes an O-ring which secures the obturator to the assembly.

5. The method of claim 1 wherein the passageway has a distal aperture through which the probe extends slightly when inserted into the brain.

6. The method of claim 1 wherein the second cannula is rotated with respect to the first cannula so that the second side-gap opens to the first side-gap thereby defining the assembly side-gap.

7. The method of claim 1 wherein the withdrawing step is performed before the separating step.

8. The method of claim 1 wherein the withdrawing step is performed before the rotating step.

9. The method of claim 1 wherein the separating step and withdrawing step are performed simultaneously.

10. The method of claim 9 wherein the proximal segment is moved through the assembly side-gap of the proximal portion before the separating and withdrawing steps are performed.

11. The method of claim 10 wherein the proximal segment is held in place while the assembly is withdrawn.

12. A method for accurately positioning a probe in a patient's brain comprising the steps of:
preparing a guide assembly having distal and proximal portions and including a first cannula, second cannula and obturator, the first cannula having a first side-gap opening to a first cavity, the second cannula having a second side-gap opening to a second cavity, the second cannula positioned within the first cavity to define an enclosed passageway having a proximal aperture and the obturator positioned in the passageway;
inserting the distal portion of the assembly into the brain, such that the proximal portion remains outside the brain;
removing the obturator from the passageway;
inserting a distal segment of the probe into the brain through the passageway such that a proximal segment of the probe remains outside the brain;
withdrawing the first cannula from the brain;
separating the first cannula from the probe;
withdrawing the second cannula from the brain; and
separating the second cannula from the probe.

13. An apparatus for accurately implanting a probe in a patient's brain comprising:
a first cannula having a first side-gap opening to a first cavity, the first side-gap having a sufficient width to allow the probe to pass therethrough; and
a second cannula having a second side-gap opening to a second cavity, the second side-gap having a sufficient width to allow the probe to pass therethrough, the second cannula receivable within the first cavity and positionable in an implantation orientation in which the first and second side-gaps are not juxtaposed, thereby providing an enclosed passageway within the apparatus with a proximal aperture for insertion of the probe, whereby the apparatus is insertable into the brain and the probe is insertable into the passageway.

14. The apparatus of claim 13 wherein the second cannula is positionable in a removal orientation in which the first and second side-gaps are juxtaposed, thereby providing an assembly side-gap through which the probe can be separated from the apparatus.

15. The apparatus of claim 14 wherein the side-gaps are slightly larger than the probe so that separation of the probe from the apparatus is facilitated.

16. The apparatus of claim 14 wherein the second cannula is rotatable within the first cannula from the implantation orientation to the removal orientation.

17. The apparatus of claim 13 further comprising an obturator having sufficient size to substantially fill the passageway so that upon insertion of the apparatus into the brain, no brain tissue enters the passageway.

18. The apparatus of claim 17 wherein the obturator includes an O-ring which secures the obturator to the cannulas.
